(19) United States
(12) Patent Application Publication
(10) Pub. No.: US 2026/0244873 A1
(43) Pub. Date: Aug. 20, 2026

(54) COMMUNICATION INTERACTION EVALUATION SYSTEM BASED ON MULTIMODAL MEDICAL-STAFF-PATIENT INTERACTION DATA FUSION AND METHOD THEREOF

(71) Applicant: TAIPEI MEDICAL UNIVERSITY, Taipei (TW)

(72) Inventor: Jen-Chieh WU, Taipei (TW)

(21) Appl. No.: 19/233,771

(22) Filed: Jun. 10, 2025

(30) Foreign Application Priority Data
Feb. 19, 2025 (TW) 114106118

(51) Int. Cl. G06F 40/35; G16H 40/20; G16H 80/00

(52) U.S. Cl. CPC G06F 40/35; G16H 40/20; G16H 80/00

(57) ABSTRACT

A communication interaction evaluation system based on multimodal medical-staff-patient interaction data fusion and a method thereof are provided. In the system, a communication interaction analysis server obtains real-time conversation speech and conversation video from a multimedia capture device, uses facial expression analysis technology, emotion recognition technology and natural language processing technology to obtain a medical-staff facial expression, a patient facial expression, a medical-staff emotion, a patient emotion, and a medical conversation analysis result; when the medical-staff facial expression and the patient facial expression are mutually-exclusive, real-time facial expression adjustment recommendation information is generated; when the medical-staff emotion and the patient emotion are mutually-exclusive, real-time emotion adjustment recommendation information is generated; or, when the medical conversation analysis result is an inappropriate conversation, real-time conversation adjustment recommendation information is generated, so that a medical staff can be provided with real-time adjustments in conversation, expression, and emotion.

### CROSS-REFERENCE STATEMENT

The present application is based on, and claims priority from, TAIWAN Patent Application Serial Number 114106118, filed Feb. 19, 2025, the disclosure of which is hereby incorporated by reference herein in its entirety.

### BACKGROUND OF THE INVENTION

### 1. Field of the Invention

The present invention is related to an evaluation system and a method thereof, and more particularly to a communication interaction evaluation system based on multimodal medical-staff-patient interaction data fusion and a method thereof.

### 2. Description of the Related Art

While nation society is entering an aging society and the average life expectancy of national continuously increasing, issues related to long-term care and death trigger medical, economic, and caregiving manpower problems. Therefore, respecting patients' medical autonomy and avoiding unnecessary medical suffering and waste of medical resources, communicating between medical staff and patients based on respect for autonomy, and maintaining professionalism, positive and proactive communication to assist patients are important. However, existing evaluation for the communication interaction between medical staff and patients is a subjective evaluation and lacks an objective evaluation.

Human emotions and reactions are very complex and varied. Besides the surface meaning, there are hidden meanings in human emotions and reactions. For example, different people may express completely different emotions and reactions when saying “I'm fine.”, for example, the person who says “I'm fine” could have genuine calmness or hide sadness. How to use human-computer interaction to analyze the communication interaction between medical staff and patients is a development direction in the medical field. If a human-computer interaction can analyze the communication interaction between medical staff and patients, the human-computer interaction can further be developed into understanding emotions and providing human-like emotional responses.

According to above-mentioned contents, what is needed is to develop an improved solution to solve the problem of lacking an objective evaluation in the communication interaction between medical staff and patients.

### SUMMARY OF THE INVENTION

An objective of the present invention is to disclose a communication interaction evaluation system based on multimodal medical-staff-patient interaction data fusion and a method thereof, to solve the problem of lacking an objective evaluation in the communication interaction between medical staff and patients.

To achieve the objective, the present invention disclose a communication interaction evaluation system based on multimodal medical-staff-patient interaction data fusion, and the communication interaction evaluation system includes a multimedia capture device, a medical prompt device, and a communication interaction analysis server. The medical prompt device, and a communication interaction analysis server includes a non-transitory computer readable storage medium, a transmission processor, a speech-to-text processor, a medical-staff-patient facial expression analysis processor, a medical-staff-patient emotion recognition processor, a medical-staff-patient conversation analysis processor, and a communication interaction analysis processor.

The multimedia capture device is configured to obtain a conversation speech and a conversation video of a conversation between a patient and medical staff in real time, each of the conversation speech and the conversation video has a timestamp. The medical prompt device is configured to receive and display at least one of real-time facial expression adjustment recommendation information, real-time emotion adjustment recommendation information and real-time conversation adjustment recommendation information to provide the medical staff with real-time adjustments in conversation, expression and emotion, and receive and display a medical-staff-patient communication interaction evaluation score within an unit time period.

The communication interaction analysis server is connected to the multimedia capture device and the medical prompt device. The non-transitory computer readable storage medium is configured to store computer readable instructions. The transmission processor is electrically connected to the non-transitory computer readable storage medium, and configured to execute the computer readable instructions to obtain the conversation speech and the conversation video from the multimedia capture device, provide at least one of the real-time facial expression adjustment recommendation information, the real-time emotion adjustment recommendation information, and the real-time conversation adjustment recommendation information to the medical prompt device, and provide the medical-staff-patient communication interaction evaluation score within the unit time period to the medical prompt device. The speech-to-text processor is electrically connected to the non-transitory computer readable storage medium, and configured to execute the computer readable instructions to use a speech-to-text technology and a voice recognition technology to convert the conversation speech into conversation text information, wherein the conversation text information is set with a timestamp corresponding to the conversation speech. The medical-staff-patient facial expression analysis processor is electrically connected to the non-transitory computer readable storage medium, and configured to execute the computer readable instructions to use a facial expression analysis technology to analyze the conversation video to obtain an medical-staff facial expression and an patient facial expression, wherein when the medical-staff facial expression and the patient facial expression are mutually-exclusive facial expressions, the medical-staff-patient facial expression analysis processor generates the real-time facial expression adjustment recommendation information. The medical-staff-patient emotion recognition processor is electrically connected to the non-transitory computer readable storage medium, and configured to execute the computer readable instructions to use an emotion recognition technology to perform an emotion recognition on the real-time conversation speech, the conversation video, and the conversation text information to obtain an medical-staff emotion and an patient emotion, wherein when the medical-staff emotion and the patient emotion are mutually-exclusive emotions, the medical-staff-patient facial expression analysis processor generates the real-time emotion adjustment recommendation information. The medical-staff-patient conversation analysis processor is electrically connected to the non-transitory computer readable storage medium, and configured to execute the computer readable instructions to use a natural language processing technology to perform a meaning-and-word analysis on medical conversation content in the conversation text information to obtain a medical conversation analysis result, wherein when the medical conversation analysis result is an inappropriate conversation, the medical-staff-patient facial expression analysis processor generates the real-time conversation adjustment recommendation information. The communication interaction analysis processor is electrically connected to the non-transitory computer readable storage medium, and configured to execute the computer readable instructions to use a communication interaction analysis model to perform an interaction mode analysis on the medical-staff facial expression, the patient facial expression, the medical-staff emotion, the patient emotion, and the medical conversation analysis result within the unit time period, to obtain the medical-staff-patient communication interaction evaluation score within the unit time period.

To achieve the objective, the present invention discloses a communication interaction evaluation method based on multimodal medical-staff-patient interaction data fusion, and the method includes steps of: obtaining a conversation speech and a conversation video from both a patient and a medical staff in real time, by a multimedia capture device, wherein each of the conversation speech and the conversation video has a timestamp; connecting a communication interaction analysis server to the multimedia capture device, obtaining the conversation speech and the conversation video from the multimedia capture device, by the communication interaction analysis server; using a speech-to-text technology and a voice recognition technology to convert the conversation speech into conversation text information, wherein the conversation text information is set with a timestamp corresponding to the conversation speech, by the communication interaction analysis server; using a facial expression analysis technology to perform facial expression analysis on the conversation video to obtain an medical-staff facial expression and an patient facial expression, by the communication interaction analysis server, wherein when the medical-staff facial expression and the patient facial expression are mutually exclusive facial expressions, the medical-staff-patient facial expression analysis processor generates real-time facial expression adjustment recommendation information; using an emotion recognition technology to perform an emotion recognition on the conversation speech, the conversation video, and the conversation text information to obtain an medical-staff emotion and an patient emotion, by the communication interaction analysis server, wherein when the medical-staff emotion and the patient emotion are mutually-exclusive emotions, the medical-staff-patient facial expression analysis processor generates real-time emotion adjustment recommendation information; using a natural language processing technology to perform a meaning-and-word analysis on the medical conversation content in the conversation text information to obtain a medical conversation analysis result, by the communication interaction analysis server, wherein when the medical conversation analysis result is an inappropriate conversation, the medical-staff-patient facial expression analysis processor generates real-time conversation adjustment recommendation information; connecting the communication interaction analysis server to a medical prompt device, to provide at least one of the real-time facial expression adjustment recommendation information, the real-time emotion adjustment recommendation information, and the real-time conversation adjustment recommendation information to the medical prompt device for display, so as to provide the medical staff with real-time adjustments in conversation, expression, and emotion; using a communication interaction analysis model to perform an interaction mode analysis on the medical-staff facial expression, the patient facial expression, the medical-staff emotion, the patient emotion, and the medical conversation analysis result within a unit time period, to obtain a medical-staff-patient communication interaction evaluation score within the unit time period, by the communication interaction analysis server; providing the medical-staff-patient communication interaction evaluation score within the unit time period, to the medical prompt device for display, by the communication interaction analysis server.

According to the above-mentioned system and method of the present invention, the communication interaction analysis server obtains real-time conversation speech and conversation video from the multimedia capture device, uses the facial expression analysis technology, the emotion recognition technology and the natural language processing technology to obtain the medical-staff facial expression, the patient facial expression, the medical-staff emotion, the patient emotion, and the medical conversation analysis result; when the medical-staff facial expression and the patient facial expression are mutually-exclusive, the real-time facial expression adjustment recommendation information is generated; when the medical-staff emotion and the patient emotion are mutually-exclusive, the real-time emotion adjustment recommendation information is generated; or, when the medical conversation analysis result is an inappropriate conversation, real-time conversation adjustment recommendation information is generated. Therefore, the medical staff can be provided with real-time adjustments in conversation, expression, and emotion; the communication interaction analysis model can perform the interaction mode analysis on all of the medical-staff facial expression, the patient facial expression, the medical-staff emotion, the patient emotion, and the medical conversation analysis result, to obtain the medical-staff-patient communication interaction evaluation score within the unit time period, and provide the medical-staff-patient communication interaction evaluation score within the unit time period to the medical-staff prompt device for display.

According to the above-mentioned solution, the present invention can achieve the technical effect of providing an objective evaluation of the communication interaction between medical staff and patients.

### BRIEF DESCRIPTION OF THE DRAWINGS

The structure, operating principle and effects of the present invention will be described in detail by way of various embodiments which are illustrated in the accompanying drawings.

FIG. 1 is a block diagram of a communication interaction evaluation system based on multimodal medical-staff-patient interaction data fusion, according to the present invention.

FIG. 2A is a schematic view of a medical-staff-patient communication interaction evaluation chart of communication interaction evaluation based on multimodal medical-staff-patient interaction data fusion, according to the present invention.

FIG. 2B is a schematic view of a patient evaluation chart of a communication interaction evaluation based on multimodal medical-staff-patient interaction data fusion, according to the present invention.

FIG. 2C is a schematic view of a medical staff evaluation chart of a communication interaction evaluation based on multimodal medical-staff-patient interaction data fusion, according to the present invention.

FIG. 3A to FIG. 3C are flowcharts of a communication interaction evaluation method based on multimodal medical-staff-patient interaction data fusion, according to the present invention.

### DETAILED DESCRIPTION OF THE EMBODIMENTS

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims.

These embodiments are provided so that this disclosure is thorough and complete, and fully conveys the inventive concept to those skilled in the art. Regarding the drawings, the relative proportions, and ratios of elements in the drawings may be exaggerated or diminished in size for the sake of clarity and convenience. Such arbitrary proportions are only illustrative and not limiting in any way. The same reference numbers are used in the drawings and description to refer to the same or like parts. As used herein, the singular forms “a”, “an” and “the” are intended to include the plural forms as well, unless the context clearly indicates otherwise.

It is to be acknowledged that, although the terms ‘first,’ ‘second,’ ‘third,’ and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the words “comprise” and “include,” and variations such as “comprises,” “comprising,” “includes,” or “including,” will be acknowledged to imply the inclusion of stated elements but not the exclusion of any other elements.

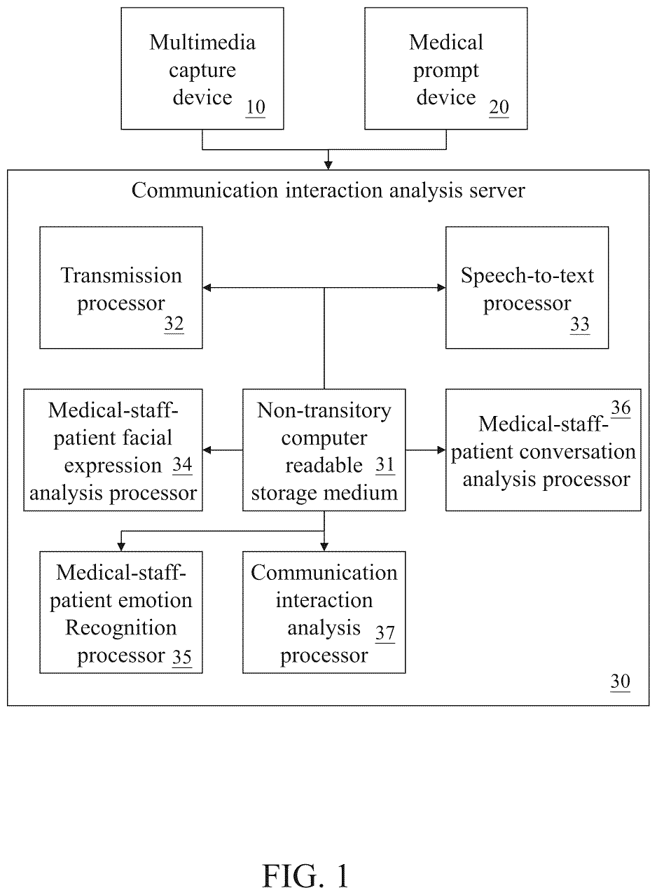

The communication interaction evaluation system based on multimodal medical-staff-patient interaction data fusion of the present invention will be illustrated in the following paragraphs. Please refer to FIG. 1. FIG. 1 is a block diagram of a communication interaction evaluation system based on multimodal medical-staff-patient interaction data fusion, according to the present invention.

As shown in FIG. 1, the communication interaction evaluation system of the present invention includes a multimedia capture device 10, a medical prompt device 20, and a communication interaction analysis server 30. The communication interaction analysis server 30 includes a non-transitory computer readable storage medium 31, a transmission processor 32, a speech-to-text processor 33, a medical-staff-patient facial expression analysis processor 34, a medical-staff-patient emotion recognition processor 35, a medical-staff-patient conversation analysis processor 36, and a communication interaction analysis processor 37.

The multimedia capture device 10 is configured to obtain a conversation speech and a conversation video from both a patient and a medical staff in real time. Each of the conversation speech and the conversation video has its own timestamp. The multimedia capture device 10 can be an integrated device that combines a microphone device and a camera device. Alternatively, the multimedia capture device 10 can be a computer device connected to the microphone device and the camera device; however, these examples are merely for exemplary illustration, and the application field of the present invention is not limited to these examples. Through the microphone device, the multimedia capture device 10 can obtain the conversation speech from the patient and the medical staff in real time, Through the camera device, the multimedia capture device 10 can obtain the conversation video from the patient and the medical staff in real time. Each of the conversation speech and the conversation video from both the patient and the medical staff has its timestamp.

The medical prompt device 20 is generally a computer with a display screen, laptop, or tablet, smartphone, but these examples are merely for exemplary illustration, and the application field of the present invention is not limited to these examples. The communication interaction analysis server 30 establishes a connection with the multimedia capture device 10 and the medical prompt device 20 through wired or wireless transmission manners, respectively. The above-mentioned wired transmission manners can be, for example, cable networks, optical fiber networks, etc.; the above-mentioned wireless transmission manners can be, for example, Wi-Fi, mobile communication networks (such as 4G, 5G, etc.); however, these examples are merely for exemplary illustration, and the application field of the present invention is not limited to these examples.

The non-transitory computer readable storage medium 31 stores computer readable instructions. The transmission processor 32, the speech-to-text processor 33, the medical-staff-patient facial expression analysis processor 34, the medical-staff-patient emotion recognition processor 35, the medical-staff-patient conversation analysis processor 36, and the communication interaction analysis processor 37 are electrically connected to the non-transitory computer readable storage medium 31 to execute the computer readable instructions.

The transmission processor 32 obtains the conversation speech and the conversation video, which can be real-time, from the multimedia capture device 10. The speech-to-text processor 33 uses a speech-to-text technology and a voice recognition technology to convert the real-time conversation speech into conversation text information. The speech-to-text technology uses acoustic models and language models to convert a speech signal into text. The conversation speech is converted into phonemes, which is the smallest unit of sound in a language, by the acoustic model. Then, the language model is used to predict the order of words corresponding to the phonemes based on context and grammatical rules, thereby improving the accuracy of the converted text. The voice recognition technology analyzes the conversation speech to extract a voiceprint feature of the medical staff and a voiceprint feature of the patient. The above-mentioned voiceprint feature can include, for example, pitch, intensity, speech rate, or formants, but these examples are merely for exemplary illustration and the application field of the present invention is not limited to these examples, By using the speech-to-text technology and the voice recognition technology, the conversation speech can be converted into the conversation text information containing the text content of the conversation between the medical staff and the patient, and the conversation text information is set with a corresponding timestamp based on the timestamp of the conversation speech.

The medical-staff-patient facial expression analysis processor 34 uses the facial expression analysis technology to perform a facial expression analysis on the real-time conversation video to obtain a medical-staff facial expression and a patient facial expression. When the medical-staff facial expression and the patient facial expression are mutually-exclusive facial expressions (that is, the medical-staff facial expression and the patient facial expression mutually exclusive), the medical-staff-patient facial expression analysis processor generates real-time facial expression adjustment recommendation information, Specifically, when the medical-staff facial expression is a “happy facial expression” and the patient facial expression is a “sorrowful facial expression”, at this time, the medical-staff facial expression and the patient facial expression are mutually-exclusive facial expressions, the medical-staff-patient facial expression analysis processor 34 generates real-time facial expression adjustment recommendation information. The real-time facial expression adjustment recommendation information can be, for example, “The patient is in a state of sadness, please treat with a serious expression.”, but these examples are merely for exemplary illustration and the application field of the present invention is not limited to these examples.

After the medical-staff-patient facial expression analysis processor 34 generates the real-time facial expression adjustment recommendation information, the real-time facial expression adjustment recommendation information can be instantly provided to the medical prompt device 20 through the transmission processor 32. The medical prompt device 20 can instantly display the real-time facial expression adjustment recommendation information after receiving real-time facial expression adjustment recommendation information from the communication interaction analysis server 30, so that the medical staff can instantly adjust their facial expressions.

The facial expression analysis technology is based on an artificial intelligence technology and a computer vision technology, the medical-staff-patient facial expression analysis processor 34 first performs a facial detection algorithm (such as Haar feature classifier, deep learning model, etc.) on the real-time conversation video to locate facial areas of the medical staff and the patient in the conversation video, marks key parts (such as the corners of the eyes, corners of the mouth, eyebrows, etc.) of the facial areas of the medical staff and the patient, and extracts geometric or appearance features representing expressions from the key parts. Next, machine learning models or deep learning models are used to map predefined expression types (such as happiness, sadness, surprise, anger, disgust, fear, etc.) based on the facial areas of the medical staff and the patient, the marked key parts, and the geometric or appearance features of the expressions.

The medical-staff-patient emotion recognition processor 35 uses an emotion recognition (or called sentiment analysis) technology to perform an emotion recognition on the real-time conversation speech, the conversation video, and the conversation text information, to obtain a medical-staff emotion and a patient emotion. When the medical-staff emotion and the patient emotion are mutually-exclusive emotions (that is, the medical-staff emotion and the patient emotion are mutually exclusive), the medical-staff-patient facial expression analysis processor generates real-time emotion adjustment recommendation information. Specifically, when the medical-staff emotion is an “excited emotion”, and the patient emotion is a “sad emotion”, the medical-staff emotion and the patient emotion are mutually-exclusive emotion, so the medical-staff-patient emotion recognition processor 35 generates real-time emotion adjustment recommendation information. The real-time emotion adjustment recommendation information can be, for example, is “The patient is in a sad emotion, being overly excited will be detrimental to the conversation between the medical staff and the patient.”, but these examples are merely for exemplary illustration, and the application field of the present invention is not limited to these examples.

When the medical-staff-patient emotion recognition processor 35 generates the real-time emotion adjustment recommendation information, the real-time emotion adjustment recommendation information is provided to the medical prompt device 20 through the transmission processor 32, the medical prompt device 20 can instantly display the real-time facial expression adjustment recommendation information when receiving real-time emotion adjustment recommendation information from the communication interaction analysis server 30, so that the medical staff can instantly adjust their emotions.

The emotion recognition technology is an artificial intelligence technology and provides recognition and analysis of individual emotions, inferring individual emotional responses by processing data such as voice, text, or expressions. The medical-staff-patient facial expression analysis processor 34 uses a natural language processing technology on the conversation text information to extract words and sentences with emotions based on vocabulary and grammar, to infer an inferred medical-staff emotion and an inferred patient emotion. In an embodiment, the medical-staff-patient facial expression analysis processor 34 infers the inferred medical-staff emotion and the inferred patient emotion from the voiceprint feature of the conversation speech. For example, the voiceprint feature being a high-pitched tone infers that the inferred medical-staff emotion or the inferred patient emotion is excitement, and the voiceprint feature being a low-pitched tone infers that the inferred medical-staff emotion or the inferred patient emotion is sadness; however, these examples are merely for exemplary illustration, and the application field of the present invention is not limited to these examples. The medical-staff-patient facial expression analysis processor 34 can call the medical-staff facial expression and the patient facial expression obtained by the medical-staff-patient facial expression analysis processor 34 and combine them with the inferred medical-staff emotion and the inferred patient emotion for comprehensive analysis to obtain an medical-staff emotion and an patient emotion.

The medical-staff-patient conversation analysis processor 36 uses the natural language processing technology to perform a meaning-and-word analysis on the medical conversation content in the conversation text information to obtain a medical conversation analysis result. When the medical conversation analysis result is an inappropriate conversation, the medical-staff-patient facial expression analysis processor generates real-time conversation adjustment recommendation information, Specifically, When the medical conversation content in the conversation text information is “You are already in the fourth stage of cancer, you can stop treatment.”, a medical conversation analysis result generated by the medical-staff-patient conversation analysis processor 36 by using the natural language processing technology to perform the meaning-and-word analysis on the medical conversation content in the conversation text information should be “inappropriate conversation”, and the medical-staff-patient conversation analysis processor 36 can generate the real-time conversation adjustment recommendation information. The real-time conversation adjustment recommendation information can be, for example, “The conversation content is too cold, it is recommended to supplement with palliative care for subsequent conversations.”, but these examples are merely for exemplary illustration and the application field of the present invention is not limited to these examples.

When the medical-staff-patient conversation analysis processor 36 generates the real-time conversation adjustment recommendation information, the real-time conversation adjustment recommendation information can be instantly provided to the medical prompt device 20 through the transmission processor 32, the medical prompt device 20 can instantly display the real-time conversation adjustment recommendation information received from the communication interaction analysis server 30, so that the medical staff can instantly adjust their conversation.

The communication interaction analysis processor 37 uses the communication interaction analysis model to perform an interaction mode analysis on all of the medical-staff facial expression, the patient facial expression, the medical-staff emotion, the patient emotion, and the medical conversation analysis result to obtain a medical-staff-patient communication interaction evaluation score within a unit time period. When the communication interaction analysis processor 37 generates the medical-staff-patient communication interaction evaluation score within the unit time period, the transmission processor 32 can provide the medical-staff-patient communication interaction evaluation score within the unit time period to the medical prompt device 20, the medical prompt device 20 can display the medical-staff-patient communication interaction evaluation score instantly when receiving the medical-staff-patient communication interaction evaluation score from the communication interaction analysis server 30, so that medical staff can know the level of communication interaction between the medical staff and the patient.

The communication interaction analysis model uses the natural language processing technology, artificial neural network (ANNs), long short-term memory (LSTM), or transformer models, but these examples are merely for exemplary illustration and the application field of the present invention is not limited to these examples, so that the communication interaction analysis model can perform emotion recognition, semantic understanding, emotional simulation, or empathetic response understanding, analyze an interaction mode between the medical staff and the patient, and score the interaction mode between the medical staff and the patient to obtain the medical-staff-patient communication interaction evaluation score.

It is worth noting that, when the medical-staff facial expression and the patient facial expression are mutually-exclusive facial expressions, the medical-staff-patient facial expression analysis processor captures a frame of the real-time conversation video as an real-time facial expression warning image based on the timestamp corresponding to the medical-staff facial expression and the patient facial expression, the real-time facial expression warning image is provided to the medical prompt device 20 through the transmission processor 32, the medical prompt device 20 can display the medical-staff-patient communication interaction evaluation score instantly when receiving the real-time facial expression warning image from the communication interaction analysis server 30, so that the medical staff can refer to the real-time facial expression warning image to adjust their expressions. Furthermore, the medical-staff-patient facial expression analysis processor 34 can mark the positions of the medical staff and the patient in the real-time facial expression warning image based on the medical-staff facial expression and the patient facial expression.

It is worth noting that, when the medical-staff emotion and the patient emotion are mutually-exclusive emotions, the medical-staff-patient emotion recognition processor 35 can capture a frame of the real-time conversation video as an real-time emotion warning image based on the timestamp corresponding to the medical-staff emotion and the patient emotion, the real-time emotion warning image is provided to the medical prompt device 20 through the transmission processor 32, the medical prompt device 20 can display the real-time emotion warning image instantly when receiving the real-time emotion warning image from the communication interaction analysis server 30, so that the medical staff can refer to the real-time emotion warning image to adjust their emotions. Furthermore, the medical-staff-patient emotion recognition processor 35 can mark the positions of the medical staff and the patient in real-time emotion warning image based on the medical-staff emotion and the patient emotion.

It is worth noting that, when the medical conversation analysis result is an inappropriate conversation, the medical-staff-patient conversation analysis processor 36 captures the real-time conversation speech as a real-time conversation warning speech based on the timestamp corresponding to the medical conversation analysis result, the real-time conversation warning speech is provided to the medical prompt device 20 through the transmission processor 32, the medical prompt device 20 can receive the real-time conversation warning speech from the communication interaction analysis server 30 and play the real-time conversation warning speech when the real-time conversation warning speech is clicked to play, so that the medical staff can refer to the real-time conversation warning speech to adjust their conversation.

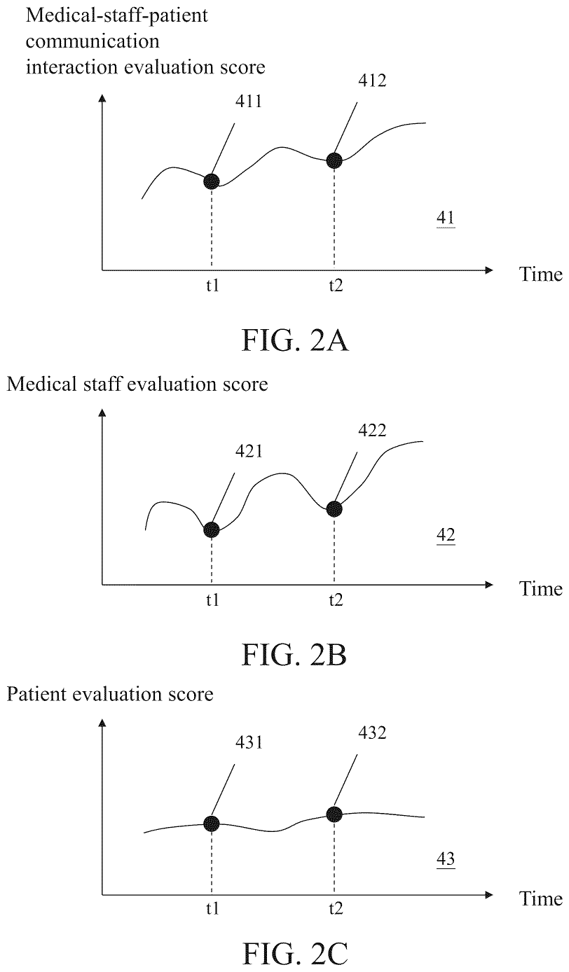

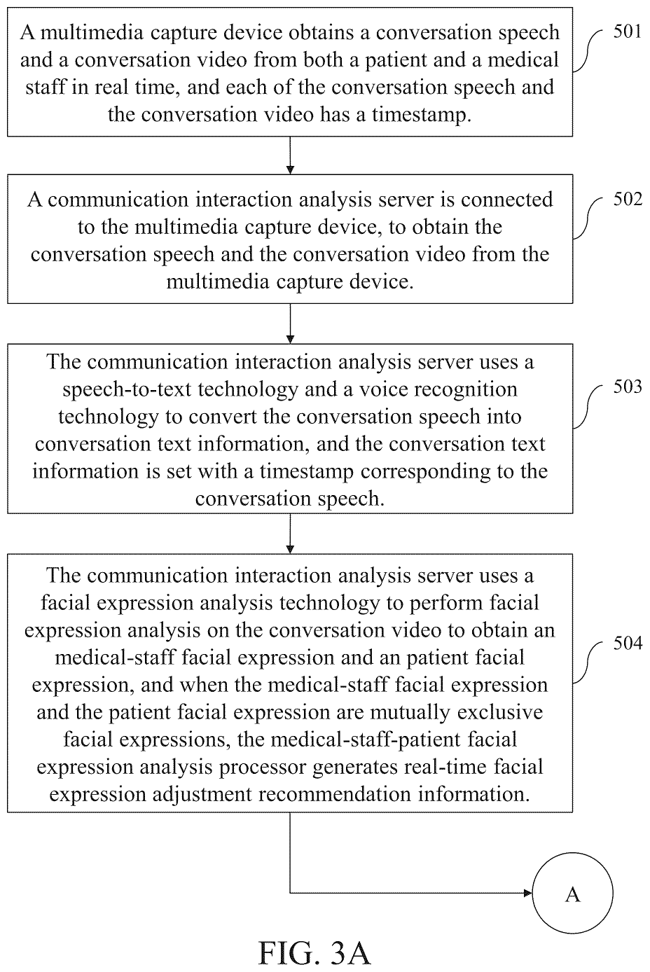

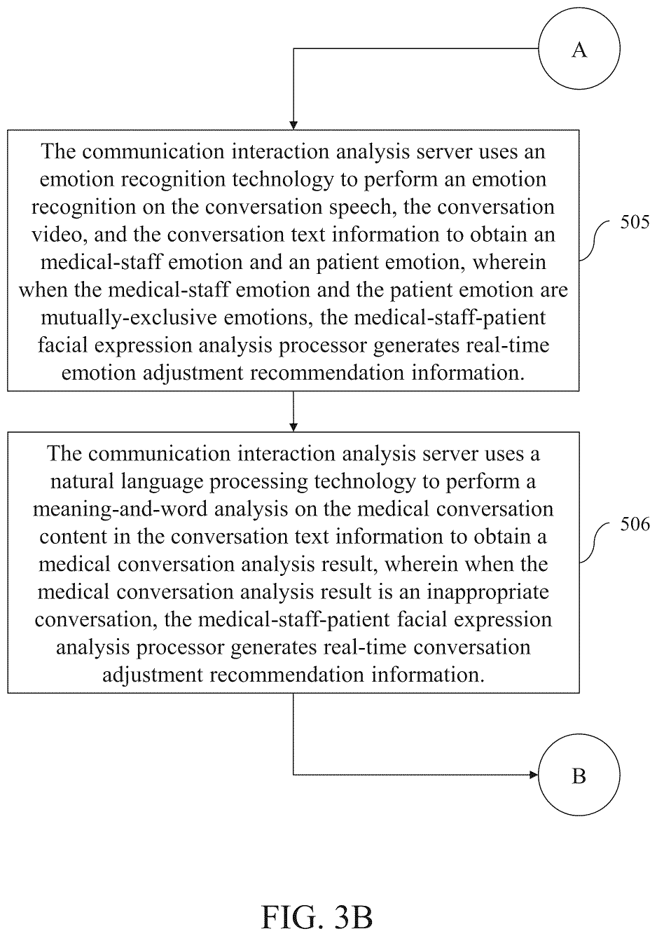

When the multimedia capture device 10 provides an end-of-conversation command to the communication interaction analysis server 30, the communication interaction analysis processor 37 uses the communication interaction analysis model to perform an interaction mode analysis on the medical-staff facial expression, patient facial expression, the medical-staff emotion, the patient emotion, the medical conversation analysis result, to obtain a total medical-staff-patient communication interaction evaluation score, and integrates the medical-staff-patient communication interaction evaluation scores corresponding to the unit time periods into a medical-staff-patient communication interaction evaluation chart 41 based on time sequence. Please refer to FIG. 2A, which shows a schematic view of the medical-staff-patient communication interaction evaluation chart 41. FIG. 2A is a schematic view of a medical-staff-patient communication interaction evaluation chart of communication interaction evaluation based on multimodal medical-staff-patient interaction data fusion, according to the present invention. The transmission processor 32 can provide the total medical-staff-patient communication interaction evaluation score and/or the medical-staff-patient communication interaction evaluation chart 41 to the medical prompt device 20, the medical prompt device 20 displays the total medical-staff-patient communication interaction evaluation score and/or the medical-staff-patient communication interaction evaluation chart 41 received from the communication interaction analysis server 30, so that the medical staff can know the level and changes of communication interaction throughout the conversation between the medical staff and the patient.

The communication interaction analysis processor 37 uses the communication interaction analysis model to perform the interaction mode analysis on the medical-staff facial expression, the medical-staff emotion, and the medical conversation analysis result within the unit time period to obtain an medical staff evaluation score within the unit time period, and then integrate the medical staff evaluation scores corresponding to the unit time periods to form a medical staff evaluation chart 42 based on time sequence, as shown in FIG. 2B, which shows a schematic view of the medical staff evaluation chart 42. FIG. 2B is a schematic view of a medical staff evaluation chart of communication interaction evaluation based on multimodal medical-staff-patient interaction data fusion, according to the present invention. The medical staff evaluation chart 42 is provided to the medical prompt device 20 through the transmission processor 32, the medical prompt device 20 can display the medical staff evaluation chart 42 received from the communication interaction analysis server 30.

The communication interaction analysis processor 37 uses the communication interaction analysis model to perform the interaction mode analysis on the patient facial expression and the patient emotion within the unit time period to obtain a patient evaluation score within a unit time period, and then integrate the patient evaluation score corresponding to the unit time periods into a patient evaluation chart 43 based on the time sequence, as shown in FIG. 2C, which shows the schematic diagram of the patient evaluation chart 43. FIG. 2C is a schematic view of a patient evaluation chart of communication interaction evaluation based on multimodal medical-staff-patient interaction data fusion, according to the present invention. The patient evaluation chart 43 is provided to the medical prompt device 20 through the transmission processor 32, and the medical prompt device 20 displays the patient evaluation chart 43 received from the communication interaction analysis server 30.

When integrating the medical-staff-patient communication interaction evaluation scores corresponding to the unit time periods into the medical-staff-patient communication interaction evaluation chart based on the time sequence, the communication interaction analysis processor 37 can mark a first warning point 411 of a first timestamp t1 and a second warning point 412 of a second timestamp t2 in the medical-staff-patient communication interaction evaluation chart 41, as shown in FIG. 2A; however, these examples are merely for exemplary illustration and the application field of the present invention is not limited to these examples. The first warning point 411 and the second warning point 412 are associated with the real-time facial expression warning image, the real-time emotion warning image, and/or the real-time conversation warning speech based on the timestamps.

When integrating the medical staff evaluation scores corresponding to the unit time periods into the medical staff evaluation chart based on the time sequence, the communication interaction analysis processor 37 can marks a first warning point 421 of the first timestamp t1 and a second warning point 422 of the second timestamp t2 in the medical staff evaluation chart 42, as shown in FIG. 2B; however, these examples are merely for exemplary illustration, and the application field of the present invention is not limited to these examples. The first warning point 421 and the second warning point 422 are associated with the real-time facial expression warning image, the real-time emotion warning image, and/or the real-time conversation warning speech based on the timestamps.

When integrating the patient evaluation scores corresponding to the unit time periods into the patient evaluation chart based on the time sequence, the communication interaction analysis processor 37 can mark a first warning point 431 of the first timestamp t1 and a second warning point 432 of the second timestamp t2 in the patient evaluation chart, as shown in FIG. 2C; however, these examples are merely for exemplary illustration, and the application field of the present invention is not limited to these examples. The first warning point 431 and the second warning point 432 are associated with the real-time facial expression warning image, the real-time emotion warning image, and/or the real-time conversation warning speech based on the timestamps.

When the first warning point 411 in the medical-staff-patient communication interaction evaluation chart 41, the first warning point 421 in the medical staff evaluation chart 42, or the first warning point 431 in the patient evaluation chart 43 displayed by the medical prompt device 20 is clicked, the medical prompt device 20 provides the timestamp corresponding to the clicked one of the first warning point 411, the first warning point 421, or the first warning point 431 to the communication interaction analysis server 30. The communication interaction analysis server 30 retrieves the corresponding the real-time facial expression warning image, the real-time emotion warning image, and/or the real-time conversation warning speech based on the received timestamp, and then provides the retrieved real-time facial expression warning image, real-time emotion warning image, and/or real-time conversation warning speech to the medical prompt device 20 for display and/or playback.

When the first warning point 411 in the medical-staff-patient communication interaction evaluation chart 41 displayed by the medical prompt device 20 is clicked, the timestamp corresponding to the first warning point 411 is t1, the first warning point 421 in the medical staff evaluation chart 42 and the first warning point 431 in the patient evaluation chart 43 corresponding to the timestamp t1 are also highlighted to provide the medical staff to know the problematic time points that medical staff should improve with a reference to the medical-staff-patient communication interaction evaluation chart 41, the medical staff evaluation chart 42, and the patient evaluation chart 43.

The communication interaction analysis processor 37 further calculates the difference between a medical-staff-patient communication interaction evaluation score of current unit time period and a medical-staff-patient communication interaction evaluation score of previous unit time period. When an absolute value of the difference is greater than or equal to an adaptive threshold, an adaptive adjustment parameter is generated based on the difference, The medical-staff-patient facial expression analysis processor 34 selects different facial detection algorithm, and/or increases or decreases the key parts based on the adaptive adjustment parameter, thereby achieving adaptive adjustment of the medical-staff-patient facial expression analysis processor 34.

The communication interaction analysis processor 37 further calculates the difference between the medical-staff-patient communication interaction evaluation score of current unit time period and the medical-staff-patient communication interaction evaluation score of previous unit time period. When the absolute value of the difference is greater than or equal to the adaptive threshold, the adaptive adjustment parameter is generated based on the difference, the medical-staff-patient emotion recognition processor 35 increases/decreases the usage data of voice, text, or expressions based on the adaptive adjustment parameter, thereby achieving the adaptive adjustment of the medical-staff-patient emotion recognition processor 35.

The communication interaction analysis processor 37 further calculates the difference between the medical-staff-patient communication interaction evaluation score of current unit time period and the medical-staff-patient communication interaction evaluation score of previous unit time period. When the absolute value of the difference is greater than or equal to the adaptive threshold, the adaptive adjustment parameter is generated based on the difference, the medical-staff-patient conversation analysis processor 36 increases/decreases the usage data of vocabulary or grammar used by the natural language processing technology based on the adaptive adjustment parameter, thereby achieving the adaptive adjustment of the medical-staff-patient conversation analysis processor 36.

It is to be particularly noted that, in actual implementation, the above-mentioned solution of the present invention can be implemented fully or partly based on hardware, for example, one or more component of the system can be implemented by hardware processor, such as integrated circuit chip, system on chip (SoC), a complex programmable logic device (CPLD), or a field programmable gate array (FPGA). The non-transitory computer-readable storage medium records computer readable program instructions, and the processor can execute the computer readable program instructions to implement concepts of the present invention. The non-transitory computer-readable storage medium can be a tangible apparatus for holding and storing the instructions executable of an instruction executing apparatus. The non-transitory computer-readable storage medium can be, but not limited to electronic storage apparatus, magnetic storage apparatus, optical storage apparatus, electromagnetic storage apparatus, semiconductor storage apparatus, or any appropriate combination thereof. More particularly, the non-transitory computer-readable storage medium can include a hard disk, an RAM memory, a read-only-memory, a flash memory, an optical disk, a floppy disc, or any appropriate combination thereof, but this exemplary list is not an exhaustive list. The non-transitory computer-readable storage medium is not interpreted as the instantaneous signal such a radio wave or other freely propagating electromagnetic wave, or electromagnetic wave propagated through waveguide, or other transmission medium (such as optical signal transmitted through fiber cable), or electric signal transmitted through electric wire. Furthermore, the computer readable program instruction can be downloaded from the non-transitory computer-readable storage medium to each calculating/processing apparatus, or downloaded through network, such as internet network, local area network, wide area network and/or wireless network, to external computer equipment or external storage apparatus. The network includes copper transmission cable, fiber transmission, wireless transmission, router, firewall, switch, hub and/or gateway. The network card or network interface of each calculating/processing apparatus can receive the computer readable program instructions from network, and forward the computer readable program instruction to store in non-transitory computer-readable storage medium of each calculating/processing apparatus. The computer readable instructions can be executed by the server 120. The computer readable instructions for executing the operations of the present invention can be assembly language instructions, instruction-set-structure instructions, machine instructions, machine-related Instructions, micro-instructions, firmware instructions, or source codes or object codes written in any combination of one or more programming languages. The programming language includes object-oriented programming languages, such as: Common Lisp, Python, C++, Objective-C, Smalltalk, Delphi, Java, Swift, C #, Perl, Ruby, or PHP; the programming language can include regular procedural programming languages, such as C language or similar programming languages.

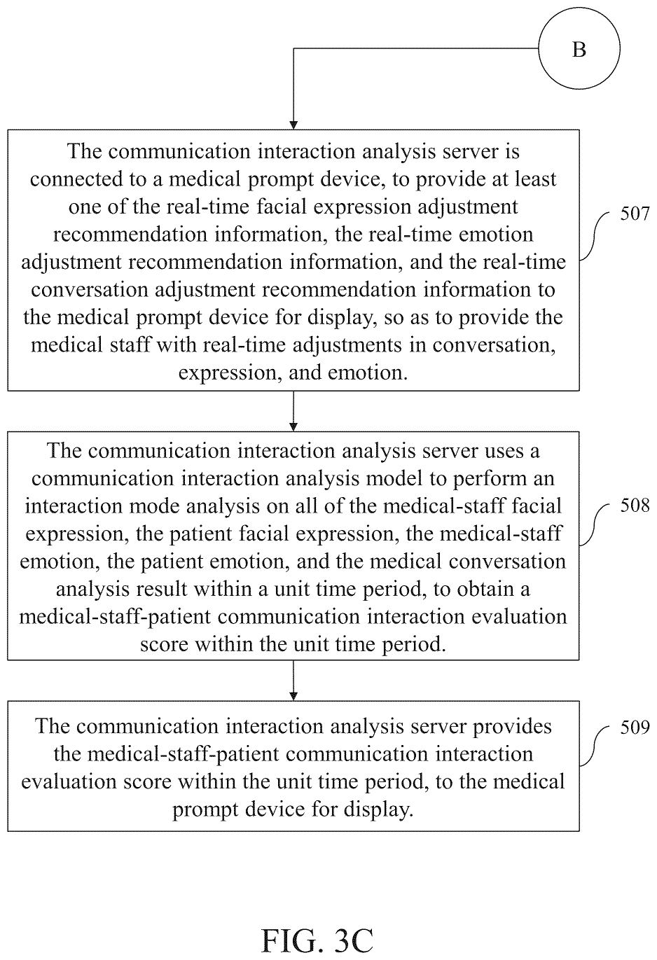

The operation of the present invention will be illustrated in the following paragraphs. Please refer to FIG. 3A to FIG. 3C, FIG. 3A to FIG. 3C are flowcharts of a communication interaction evaluation method based on multimodal medical-staff-patient interaction data fusion, according to the present invention.

As shown in FIG. 3A to FIG. 3C, the communication interaction evaluation method based on multimodal medical-staff-patient interaction data fusion disclosed in the present invention, includes the following steps.

In a step 501, a multimedia capture device obtains a conversation speech and a conversation video from both a patient and a medical staff in real time, and each of the conversation speech and the conversation video has a timestamp. In a step 502, the communication interaction analysis server is connected to the multimedia capture device, to obtain the conversation speech and the conversation video from the multimedia capture device. In a step 503, the communication interaction analysis server uses a speech-to-text technology and a voice recognition technology to convert the conversation speech into conversation text information, and the conversation text information is set with a timestamp corresponding to the conversation speech. In a step 504, the communication interaction analysis server uses a facial expression analysis technology to perform facial expression analysis on the conversation video to obtain an medical-staff facial expression and an patient facial expression, and when the medical-staff facial expression and the patient facial expression are mutually exclusive facial expressions, the medical-staff-patient facial expression analysis processor generates real-time facial expression adjustment recommendation information. In a step 505, the communication interaction analysis server uses an emotion recognition technology to perform an emotion recognition on the conversation speech, the conversation video, and the conversation text information to obtain an medical-staff emotion and an patient emotion, wherein when the medical-staff emotion and the patient emotion are mutually-exclusive emotions, the medical-staff-patient facial expression analysis processor generates real-time emotion adjustment recommendation information. In a step 506, the communication interaction analysis server uses a natural language processing technology to perform a meaning-and-word analysis on the medical conversation content in the conversation text information to obtain a medical conversation analysis result, wherein when the medical conversation analysis result is an inappropriate conversation, the medical-staff-patient facial expression analysis processor generates real-time conversation adjustment recommendation information. In a step 507, the communication interaction analysis server is connected to a medical prompt device, to provide at least one of the real-time facial expression adjustment recommendation information, the real-time emotion adjustment recommendation information, and the real-time conversation adjustment recommendation information to the medical prompt device for display, so as to provide the medical staff with real-time adjustments in conversation, expression, and emotion. In a step 508, the communication interaction analysis server uses a communication interaction analysis model to perform an interaction mode analysis on all of the medical-staff facial expression, the patient facial expression, the medical-staff emotion, the patient emotion, and the medical conversation analysis result within a unit time period, to obtain a medical-staff-patient communication interaction evaluation score within the unit time period. In a step 509, the communication interaction analysis server provides the medical-staff-patient communication interaction evaluation score within the unit time period, to the medical prompt device for display.

According to above-mentioned contents, the communication interaction analysis server obtains real-time conversation speech and conversation video from the multimedia capture device, uses the facial expression analysis technology, the emotion recognition technology and the natural language processing technology to obtain the medical-staff facial expression, the patient facial expression, the medical-staff emotion, the patient emotion, and the medical conversation analysis result; when the medical-staff facial expression and the patient facial expression are mutually-exclusive, the real-time facial expression adjustment recommendation information is generated; when the medical-staff emotion and the patient emotion are mutually-exclusive, the real-time emotion adjustment recommendation information is generated; or, when the medical conversation analysis result is an inappropriate conversation, real-time conversation adjustment recommendation information is generated. Therefore, the medical staff can be provided with real-time adjustments in conversation, expression, and emotion; the communication interaction analysis model can perform the interaction mode analysis on all of the medical-staff facial expression, the patient facial expression, the medical-staff emotion, the patient emotion, and the medical conversation analysis result, to obtain the medical-staff-patient communication interaction evaluation score within the unit time period, and provide the medical-staff-patient communication interaction evaluation score within the unit time period to the medical-staff prompt device for display.

According to the above-mentioned solution, the present invention can solve the problem of lacking an objective evaluation in the communication interaction between medical staff and patients and achieve the technical effect of providing an objective evaluation of the communication interaction between medical staff and patients.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A communication interaction evaluation system based on multimodal medical-staff-patient interaction data fusion, comprising:
a multimedia capture device, configured to obtain a conversation speech and a conversation video of a conversation between a patient and medical staff in real time, each of the conversation speech and the conversation video has a timestamp;
a medical prompt device, configured to receive and display at least one of real-time facial expression adjustment recommendation information, real-time emotion adjustment recommendation information and real-time conversation adjustment recommendation information to provide the medical staff with real-time adjustments in conversation, expression and emotion, and receive and display a medical-staff-patient communication interaction evaluation score within an unit time period;
a communication interaction analysis server, connected to the multimedia capture device and the medical prompt device, and comprising:
a non-transitory computer readable storage medium, configured to store computer readable instructions;
a transmission processor, electrically connected to the non-transitory computer readable storage medium, and configured to execute the computer readable instructions to obtain the conversation speech and the conversation video from the multimedia capture device, provide at least one of the real-time facial expression adjustment recommendation information, the real-time emotion adjustment recommendation information, and the real-time conversation adjustment recommendation information to the medical prompt device, and provide the medical-staff-patient communication interaction evaluation score within the unit time period to the medical prompt device;
a speech-to-text processor, electrically connected to the non-transitory computer readable storage medium, and configured to execute the computer readable instructions to use a speech-to-text technology and a voice recognition technology to convert the conversation speech into conversation text information, wherein the conversation text information is set with a timestamp corresponding to the conversation speech;
a medical-staff-patient facial expression analysis processor, electrically connected to the non-transitory computer readable storage medium, and configured to execute the computer readable instructions to use a facial expression analysis technology to analyze the conversation video to obtain an medical-staff facial expression and an patient facial expression, wherein when the medical-staff facial expression and the patient facial expression are mutually-exclusive facial expressions, the medical-staff-patient facial expression analysis processor generates the real-time facial expression adjustment recommendation information;
a medical-staff-patient emotion recognition processor, electrically connected to the non-transitory computer readable storage medium, and configured to execute the computer readable instructions to use an emotion recognition technology to perform an emotion recognition on the real-time conversation speech, the conversation video, and the conversation text information to obtain an medical-staff emotion and an patient emotion, wherein when the medical-staff emotion and the patient emotion are mutually-exclusive emotions, the medical-staff-patient facial expression analysis processor generates the real-time emotion adjustment recommendation information;
a medical-staff-patient conversation analysis processor, electrically connected to the non-transitory computer readable storage medium, and configured to execute the computer readable instructions to use a natural language processing technology to perform a meaning-and-word analysis on medical conversation content in the conversation text information to obtain a medical conversation analysis result, wherein when the medical conversation analysis result is an inappropriate conversation, the medical-staff-patient facial expression analysis processor generates the real-time conversation adjustment recommendation information; and
a communication interaction analysis processor, electrically connected to the non-transitory computer readable storage medium, and configured to execute the computer readable instructions to use a communication interaction analysis model to perform an interaction mode analysis on the medical-staff facial expression, the patient facial expression, the medical-staff emotion, the patient emotion, and the medical conversation analysis result within the unit time period, to obtain the medical-staff-patient communication interaction evaluation score within the unit time period.

2. The communication interaction evaluation system based on multimodal medical-staff-patient interaction data fusion according to claim 1, wherein the communication interaction analysis processor uses the communication interaction analysis model to perform the interaction mode analysis on all of the medical-staff facial expressions, the patient facial expressions, the medical-staff emotions, the patient emotions, and the medical conversation analysis results, to obtain a total medical-staff-patient communication interaction evaluation score, and integrates the medical-staff-patient communication interaction evaluation scores corresponding to the unit time periods to form a medical-staff-patient communication interaction evaluation chart based on time sequence, the transmission processor provides at least one of the total medical-staff-patient communication interaction evaluation score and the medical-staff-patient communication interaction evaluation chart to the medical prompt device for display.

3. The communication interaction evaluation system based on multimodal medical-staff-patient interaction data fusion according to claim 1, wherein the communication interaction analysis processor further calculates a difference between a medical-staff-patient communication interaction evaluation score of a current unit time period and a medical-staff-patient communication interaction evaluation score of a previous unit time period, when an absolute value of the difference is greater than or equal to an adaptive threshold, an adaptive adjustment parameter is generated based on the difference, and the medical-staff-patient facial expression analysis processor performs adaptive adjustment based on the adaptive adjustment parameter,
wherein the communication interaction analysis processor further calculates the difference between the medical-staff-patient communication interaction evaluation score of the current unit time period and the medical-staff-patient communication interaction evaluation score of the previous unit time period, when the absolute value of the difference is greater than or equal to the adaptive threshold, the adaptive adjustment parameter is generated based on the difference, and the medical-staff-patient emotion recognition processor performs adaptive adjustment based on the adaptive adjustment parameter,
wherein the communication interaction analysis processor further calculates the difference between the medical-staff-patient communication interaction evaluation score of the current unit time period and the medical-staff-patient communication interaction evaluation score of the previous unit time period, when the absolute value of the difference is greater than or equal to the adaptive threshold, the adaptive adjustment parameter is generated based on the difference, and the medical-staff-patient conversation analysis processor performs adaptive adjustment based on the adaptive adjustment parameter.

4. The communication interaction evaluation system based on multimodal medical-staff-patient interaction data fusion according to claim 1, wherein the communication interaction analysis processor uses the communication interaction analysis model to perform an interaction mode analysis on the medical-staff facial expression, the medical-staff emotion, and the medical conversation analysis result within the unit time period, to obtain an medical staff evaluation score within the unit time period, and integrates the medical staff evaluations scores corresponding to the unit time periods to form a medical staff evaluation chart based on time sequence, and the transmission processor provides the medical staff evaluation chart to the medical prompt device for display,
wherein the communication interaction analysis processor uses the communication interaction analysis model to perform the interaction mode analysis on the patient facial expressions and the patient emotions within the unit time period, to obtain an patient evaluation score within the unit time period, and integrates the patient evaluation scores corresponding to the unit time periods to form a patient evaluation chart based on time sequence, and the transmission processor provides the patient evaluation chart to the medical prompt device for display.

5. The communication interaction evaluation system based on multimodal medical-staff-patient interaction data fusion according to claim 1, wherein when the medical-staff facial expression and the patient facial expression are the mutually-exclusive facial expressions, the medical-staff-patient facial expression analysis processor captures a frame of the conversation video as an real-time facial expression warning image based on the timestamp corresponding to the medical-staff facial expression and the patient facial expression,
wherein when the medical-staff emotion and the patient emotion are the mutually-exclusive emotions, the medical-staff-patient emotion analysis processor is configured to capture a frame of the conversation video as a real-time emotion warning image based on the timestamp corresponding to the medical-staff emotion and the patient emotion;
wherein when the medical conversation analysis result is the inappropriate conversation, the medical-staff-patient conversation analysis processor captures the conversation speech as a real-time conversation warning speech based on the timestamp corresponding to the medical conversation analysis result.

6. A communication interaction evaluation method based on multimodal medical-staff-patient interaction data fusion, comprising:
obtaining a conversation speech and a conversation video from both a patient and a medical staff in real time, by a multimedia capture device, wherein each of the conversation speech and the conversation video has a timestamp;
connecting a communication interaction analysis server to the multimedia capture device, and obtaining the conversation speech and the conversation video from the multimedia capture device, by the communication interaction analysis server;
using a speech-to-text technology and a voice recognition technology to convert the conversation speech into conversation text information, wherein the conversation text information is set with a timestamp corresponding to the conversation speech, by the communication interaction analysis server;
using a facial expression analysis technology to perform facial expression analysis on the conversation video to obtain an medical-staff facial expression and an patient facial expression, by the communication interaction analysis server, wherein when the medical-staff facial expression and the patient facial expression are mutually exclusive facial expressions, the medical-staff-patient facial expression analysis processor generates real-time facial expression adjustment recommendation information;
using an emotion recognition technology to perform an emotion recognition on the conversation speech, the conversation video, and the conversation text information to obtain an medical-staff emotion and an patient emotion, by the communication interaction analysis server, wherein when the medical-staff emotion and the patient emotion are mutually-exclusive emotions, the medical-staff-patient facial expression analysis processor generates real-time emotion adjustment recommendation information;
using a natural language processing technology to perform a meaning-and-word analysis on the medical conversation content in the conversation text information to obtain a medical conversation analysis result, by the communication interaction analysis server, wherein when the medical conversation analysis result is an inappropriate conversation, the medical-staff-patient facial expression analysis processor generates real-time conversation adjustment recommendation information;
connecting the communication interaction analysis server to a medical prompt device, to provide at least one of the real-time facial expression adjustment recommendation information, the real-time emotion adjustment recommendation information, and the real-time conversation adjustment recommendation information to the medical prompt device for display, so as to provide the medical staff with real-time adjustments in conversation, expression, and emotion;
using a communication interaction analysis model to perform an interaction mode analysis on the medical-staff facial expression, the patient facial expression, the medical-staff emotion, the patient emotion, and the medical conversation analysis result within a unit time period, to obtain a medical-staff-patient communication interaction evaluation score within the unit time period, by the communication interaction analysis server; and
providing the medical-staff-patient communication interaction evaluation score within the unit time period, to the medical prompt device for display, by the communication interaction analysis server.

7. The communication interaction evaluation method based on multimodal medical-staff-patient interaction data fusion according to claim 6, wherein the communication interaction analysis processor uses the communication interaction analysis model to perform the interaction mode analysis on all of the medical-staff facial expressions, the patient facial expressions, the medical-staff emotions, the patient emotions, and the medical conversation analysis results, to obtain a total medical-staff-patient communication interaction evaluation score, and integrates the medical-staff-patient communication interaction evaluation scores corresponding to the unit time periods to form a medical-staff-patient communication interaction evaluation chart based on time sequence, the transmission processor provides at least one of the total medical-staff-patient communication interaction evaluation score and the medical-staff-patient communication interaction evaluation chart to the medical prompt device for display.

8. The communication interaction evaluation method based on multimodal medical-staff-patient interaction data fusion according to claim 6, wherein the communication interaction analysis processor further calculates a difference between a medical-staff-patient communication interaction evaluation score of a current unit time period and a medical-staff-patient communication interaction evaluation score of a previous unit time period, when an absolute value of the difference is greater than or equal to an adaptive threshold, an adaptive adjustment parameter is generated based on the difference, and the medical-staff-patient facial expression analysis processor performs adaptive adjustment based on the adaptive adjustment parameter,
wherein the communication interaction analysis processor further calculates the difference between the medical-staff-patient communication interaction evaluation score of the current unit time period and the medical-staff-patient communication interaction evaluation score of the previous unit time period, when the absolute value of the difference is greater than or equal to the adaptive threshold, the adaptive adjustment parameter is generated based on the difference, and the medical-staff-patient emotion recognition processor performs adaptive adjustment based on the adaptive adjustment parameter,
wherein the communication interaction analysis processor further calculates the difference between the medical-staff-patient communication interaction evaluation score of the current unit time period and the medical-staff-patient communication interaction evaluation score of the previous unit time period, when the absolute value of the difference is greater than or equal to the adaptive threshold, the adaptive adjustment parameter is generated based on the difference, and the medical-staff-patient conversation analysis processor performs adaptive adjustment based on the adaptive adjustment parameter.

9. The communication interaction evaluation method based on multimodal medical-staff-patient interaction data fusion according to claim 6, wherein the communication interaction analysis processor uses the communication interaction analysis model to perform an interaction mode analysis on the medical-staff facial expression, the medical-staff emotion, and the medical conversation analysis result within the unit time period, to obtain an medical staff evaluation score within the unit time period, and integrates the medical staff evaluations scores corresponding to the unit time periods to form a medical staff evaluation chart based on time sequence, and the transmission processor provides the medical staff evaluation chart to the medical prompt device for display,
wherein the communication interaction analysis processor uses the communication interaction analysis model to perform the interaction mode analysis on the patient facial expressions and the patient emotions within the unit time period, to obtain an patient evaluation score within the unit time period, and integrates the patient evaluation scores corresponding to the unit time periods to form a patient evaluation chart based on time sequence, and the transmission processor provides the patient evaluation chart to the medical prompt device for display.

10. The communication interaction evaluation method based on multimodal medical-staff-patient interaction data fusion according to claim 6, wherein when the medical-staff facial expression and the patient facial expression are the mutually-exclusive facial expressions, the medical-staff-patient facial expression analysis processor captures a frame of the conversation video as an real-time facial expression warning image based on the timestamp corresponding to the medical-staff facial expression and the patient facial expression,
wherein when the medical-staff emotion and the patient emotion are the mutually-exclusive emotions, the medical-staff-patient emotion analysis processor is configured to capture a frame of the conversation video as an real-time emotion warning image based on the timestamp corresponding to the medical-staff emotion and the patient emotion; wherein when the medical conversation analysis result is the inappropriate conversation, the medical-staff-patient conversation analysis processor captures the conversation speech as a real-time conversation warning speech based on the timestamp corresponding to the medical conversation analysis result.
